SEQUENCE LISTING (17 sequences)

SEQ ID NO: 1 - protein, 12 aa, Homo sapiens
THRPPMWSPVWP

SEQ ID NO: 2 - protein, 13 aa, Homo sapiens
HLNILSTLWKYRC

SEQ ID NO: 3 - protein, 13 aa, Homo sapiens
TGNYKALHPHNGC

SEQ ID NO: 4 - protein, 18 aa, Homo sapiens
CNCKAPETALCARRCQQH

SEQ ID NO: 5 - protein, 14 aa, Homo sapiens
PTVIHGKREVTLHL

SEQ ID NO: 6 - protein, 20 aa, Homo sapiens
ELKHFEAKIEKHNHYQKQLE

SEQ ID NO: 7 - protein, 19 aa, Homo sapiens
TFFYGGSRGKRNNFKTEEY

SEQ ID NO: 8 - protein, 15 aa, Homo sapiens
GGGGYGRKKRRQRRR

SEQ ID NO: 9 - protein, 18 aa, Homo sapiens
RGGRLSYSRRRFSTSTGR

SEQ ID NO: 10 - protein, 31 aa, Homo sapiens
YQQVLTSLPSQNVLQIANDLENLRDLLHLLC

SEQ ID NO: 11 - protein, 14 aa, Homo sapiens
KKRTLRKNDRKKRC

SEQ ID NO: 12 - protein, 39 aa, Homo sapiens
SSVIDALQYKLEGTTRLTRKRGLKLATALSLSNKFVEGS

SEQ ID NO: 13 - protein, 29 aa, Homo sapiens
YTIWMPENPRPGTPCDIFTNSRGKRASNG

SEQ ID NO: 14 - protein, 7 aa, Homo sapiens
HAIYPRH

SEQ ID NO: 15 - protein, 9 aa, Homo sapiens
CRTIGPSVC

SEQ ID NO: 16 - protein, 4 aa, Homo sapiens
CAQK

SEQ ID NO: 17 - protein, 14 aa, Homo sapiens
SACPSHLTKMCGGG

(19) United States
(12) Patent Application Publication
(10) Pub. No.: US 2025/0090673 A1
(43) Pub. Date: Mar. 20, 2025

(54) CONJUGATE FOR TARGETING CENTRAL NERVOUS SYSTEM

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US)

(21) Appl. No.: 18/967,383

(22) Filed: Dec. 3, 2024

Related U.S. Application Data
(63) Continuation of application No. PCT/US2023/067836, filed on Jun. 2, 2023.
(60) Provisional application No. 63/349,302, filed on Jun. 6, 2022.

(51) Int. Cl. A61K 47/64; A61K 9/51; A61K 31/704; A61K 47/59; A61K 47/60; C07K 16/28; C07K 16/32

(52) U.S. Cl. CPC A61K 47/64; A61K 9/5123; A61K 9/5146; A61K 9/5153; A61K 31/704; A61K 47/593; A61K 47/60; C07K 16/2809; C07K 16/32

(57) ABSTRACT

The present invention provides a conjugate comprising: (a) a CNS-targeting ligand, (b) a hydrophilic polymer of polyethylene glycol (PEG), polylactic acid (PLA), polylactic-co-glycolic acid (PLGA), or dextran, and (c) a flavonoid. The present invention also provides to a micelle nanoparticle composition comprising: (a) an outer shell comprising the conjugate, optionally (b) an inner shell comprising oligomeric (−)-epigallocatechin gallate (OEGCG), and optionally (c) a CNS disease-treating molecule encapsulated in the inner shell. The present invention further provides a method for treating a CNS disease by administering an effective amount of the present nanoparticle composition to a subject. The CNS-targeting ligand targets the CNS tissue and delivers active ingredients to CNS tissue for treating the CNS pathogenic conditions.

### CROSS-REFERENCE TO RELATED APPLICATION(S)

This application is a continuation of PCT/US2023/067836, filed Jun. 2, 2023; which claims the benefit of U.S. Provisional Application No. 63/349,302, filed Jun. 6, 2022. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on Jun. 1, 2023, is named SequenceListing.xml and is 15300 kilobytes in size.

### FIELD OF THE INVENTION

The present invention relates to a conjugate comprising: (a) a CNS (central nervous system) targeting ligand, (b) a hydrophilic polymer of polyethylene glycol (PEG), polylactic acid (PLA), polylactic-co-glycolic acid (PLGA), or dextran, and (c) a flavonoid, wherein the hydrophilic polymer covalently binds to the flavonoid and the CNS-targeting ligand. The present invention relates to micelle nanoparticles comprising: (a) an outer shell comprising a CNS-targeting ligand-hydrophilic polymer-(−)-epigallocatechin gallate (EGCG) conjugate, optionally (b) inner shell comprising oligomeric (OEGCG), and optionally (c) a CNS treating agent encapsulated in the inner shell.

### BACKGROUND OF THE INVENTION

Central nervous system (CNS) diseases are a group of neurological disorders that affect the structure or function of the brain or spinal cord, which collectively form the CNS. The condition may be an inherited metabolic disorder, the result of neural damages from infections, neurodegenerative conditions, stroke, brain tumor or other problems from unknown or multiple factors. CNS diseases include brain tumors, neurodegenerative diseases such as amyotrophic lateral sclerosis, multiple sclerosis, Parkinson's disease, Alzheimer's disease, Huntington's disease, multiple system atrophy, and prion diseases, migraine, infection, addiction, arachnoid cysts, attention deficit/hyperactivity disorder (ADHD), autism, catalepsy, encephalitis, epilepsy/seizures, infection, locked-in syndrome, meningitis, migraine, myelopathy, Tourette's syndrome.

A CNS tumor occurs when abnormal cells form within the brain and the spinal cord. Brain tumors include malignant tumors and benign (non-cancerous) tumors. These can be further classified as primary tumors, which start within the brain, and secondary tumors, which most commonly have spread from tumors located outside the brain. Spinal tumors are neoplasms located in either the vertebral column or the spinal cord. Brain tumor can be primary cancer of the brain, or metastatic cancer to the brain.

Stroke is a medical condition in which poor blood flow to the brain causes cell death. There are two main types of strokes: ischemic, due to lack of blood flow, and hemorrhagic, due to bleeding. The main risk factor for stroke is high blood pressure. Other risk factors include high blood cholesterol, tobacco smoking, obesity, diabetes mellitus, a previous transient ischemic attack, end-stage kidney disease, and atrial fibrillation. There is no therapy to regenerate dead brain cells after stroke. Treatment for stroke is very limited, one FDA-approved drug for ischemic stroke is the tissue plasminogen activator (tPA) that breaks down blood clots in the brain. Prevention is an important clinical strategy. Oral anticoagulants such as warfarin are the mainstay of stroke prevention.

Neurodegenerative diseases are a group of diseases which primarily affect the neurons in the human brain. Examples of neurodegenerative diseases are Alzheimer's disease (AD) and other dementias, Parkinson's disease (PD) and Parkinsonism, prion disease, motor neurone diseases (MND), Huntington's disease (HD), spinocerebellar ataxia (SCA) and spinal muscular atrophy (SMA). Some neurodegenerative disorders are caused by inherited genetic changes. Most neurodegenerative disorders are due to a combination of genetic and environmental factors. This makes it difficult to predict who will develop the disease. Two major neurodegenerative diseases are Alzheimer's and Parkinson's diseases.

Alzheimer's disease (AD) is a neurodegenerative disease that usually starts slowly and progressively worsens. As the disease advances, symptoms can include problems with language, disorientation (including easily getting lost), mood swings, loss of motivation, self-neglect, and behavioral issues. Alzheimer's disease is believed to occur when abnormal amounts of amyloid beta (Aβ), accumulating extracellularly as amyloid plaques and tau proteins, or intracellularly as neurofibrillary tangles, form in the brain, affecting neuronal functioning and connectivity, resulting in a progressive loss of brain function. Currently, no treatments stop or reverse its progression, though some may temporarily improve symptoms.

Parkinson's disease (PD) is a long-term degenerative disorder of the central nervous system that mainly affects the motor system. It is sometimes referred to as a type of neurodegenerative disease called synucleinopathy due to an abnormal accumulation of the protein alpha-synuclein in the brain. The most obvious early symptoms of PD are tremor, rigidity, slowness of movement, and difficulty with walking. Cognitive and behavioral problems may also occur with depression, anxiety, and apathy occurring in many people with PD. Parkinson's disease dementia becomes common in the advanced stages of the disease. No cure for PD is known; treatment aims to reduce the effects of the symptoms.

Drug delivery to the brain is the process of passing therapeutically active molecules across the blood-brain barrier for the purpose of treating brain maladies. This is a complex process that must consider the complex anatomy of the brain as well as the restrictions imposed by the special junctions of the blood-brain barrier.

The blood-brain barrier is formed by special tight junctions between endothelial cells lining brain blood vessels. Blood vessels of all tissues contain this monolayer of endothelial cells, however only brain endothelial cells have tight junctions preventing passive diffusion of most substances into the brain tissue.

The blood-brain barrier (BBB) is a highly selective semipermeable border of endothelial cells of the central nervous system (CNS) that prevents solutes in the circulating blood vessel from non-selectively crossing into central nervous system where neurons reside. Therefore, BBB is a barrier hindering effective drugs from accumulating at brain. There are 4 pathways for BBB penetration (passive diffusion, carrier-mediated transport, receptor-mediated transcytosis and adsorptive-mediated transcytosis). Adsorptive-mediated transcytosis (AMT) is the major pathway. AMT pathway uses Caveolae as transporting vehicle. Caveolae are a subgroup of lipid rafts present in endothelial cells of BBB. Caveolae-mediated endocytosis is a critical transporting mechanism for macromolecule uptake from blood stream to CNS.

For many therapeutic agents, only a small portion of the medication reaches the tissue to be affected, for example, in chemotherapy where roughly 99% of the drugs administered do not reach the tumor site. Targeted drug delivery seeks to concentrate the medication in the tissues of interest while reducing the relative concentration of the medication in the remaining tissues. For example, by avoiding the host's defense mechanisms and inhibiting non-specific distribution in the liver and spleen, a system can reach the intended site of action in higher concentrations. Targeted delivery is believed to improve efficacy while reducing side-effects.

### BRIEF DESCRIPTION OF THE DRAWINGS

FIG. 1 shows one micelle composition of the present invention, in which a drug molecule is encapsulated within the micelle, and the micelle comprises Ligand-PEG-EGCG conjugate in an outer shell and oligomeric EGCG (OEGCG) in an inner shell.

FIG. 2 shows one micelle composition of the present invention, in which a drug molecule is encapsulated within the micelle, and the micelle comprises Ligand-PEG-EGCG conjugate plus bare PEG-EGCG in an outer shell and oligomeric EGCG (OEGCG) in an inner shell.

FIG. 3 shows the chemical synthesis scheme of TfR-PEG-EGCG via conjugating the N-terminal of TfR peptide to HOOC-PEG-EGCG.

FIG. 4 shows the chemical synthesis scheme of Tet1-PEG-EGCG via conjugating the N-terminal of Tet1 peptide to HOOC-PEG-EGCG.

FIG. 5 shows the chemical synthesis scheme of TC13-PEG-EGCG via conjugating the N-terminal of TC13 peptide to HOOC-PEG-EGCG.

FIG. 6 shows the successful formulation of TfR-MINC-doxorubicin (A), Tet1-MINC-doxorubicin (B) and TC13-MINC-doxorubicin (C).

FIG. 7 shows the brain endothelial cell uptake of TfR-MINC-doxorubicin, Tet1-MINC-doxorubicin and TC13-MINC-doxorubicin by measuring fluorescence signals.

FIG. 8 shows the chemical synthesis scheme of TfR-PEG-EGCG via conjugating the C-terminal of TfR peptide to HO-PEG-EGCG.

FIG. 9 shows the chemical synthesis scheme of Tet1-PEG-EGCG via conjugating the C-terminal of Tet1 peptide to HO-PEG-EGCG.

FIG. 10 shows the chemical synthesis scheme of TC13-PEG-EGCG via conjugating the C-terminal of TC13 peptide to HO-PEG-EGCG.

FIG. 11 shows the chemical synthesis scheme of adenosine-PEG-EGCG via conjugating the primary OH group of adenosine to HOOC-PEG-EGCG.

FIG. 12 shows the chemical synthesis scheme of TfR-PLA-EGCG via conjugating the N-terminal of TfR peptide to HOOC-PLA-EGCG.

FIG. 13 shows the chemical synthesis scheme of TfR-PLGA-EGCG via conjugating the N-terminal of TfR peptide to HOOC-PLGA-EGCG.

FIG. 14 shows the chemical synthesis scheme of TfR-Dextran-EGCG via conjugating the C-terminal of TfR peptide to HO-Dextran-EGCG.

### DETAILED DESCRIPTION OF THE INVENTION

### Definitions

The term “about” is defined as ±10%, preferably ±5%, of the recited value.

The term “a CNS-targeting ligand”, as used herein, refers to a molecule of molecular weight<10000 Daltons, for example, 300-3500 Daltons, such as a peptide or a small molecule, that binds or targets receptors on a CNS cell surface or CNS environment.

The term “cytokines” refer to proteins (˜5-70 kDa) important in cell signaling. Cytokines have been shown to be involved in autocrine, paracrine, and endocrine signaling as immunomodulating agents. Cytokines include interferons, interleukins, lymphokines, tumor necrosis factors, and chemokines.

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 2-50, 3-50, or 3-20 monomers of EGCG that are covalently linked. OEGCG preferably contains 4 to 12 monomers of EGCG.

The term “nanoparticles” refers to particles with a diameter below 1 μm and between 1-999 nm.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The term “MINC” (Multi-pathway Immune-modulating Nanocomplex Combination therapy) is a platform technology. As used in this application, MINC utilizes the bioactivity of PEG-flavonoid conjugate and oligomeric EGCG (OEGCG). MINC can encapsulate additional CNS treating agents to form MINC-agent.

The term “MINC-agent”, as used in this application, is a micelle with a shell formed by CNS-targeting ligand-PEG-flavonoid conjugate and optionally oligomeric flavonoid such as OEGCG and has an agent encapsulated within the shell.

Unless otherwise specified, “%” as used in this application, refers to weight %.

### Flavonoids

Flavonoids suitable for the present invention have the general structure of Formula I:

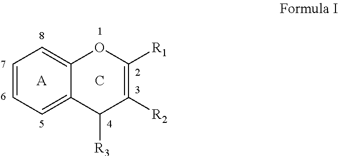

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

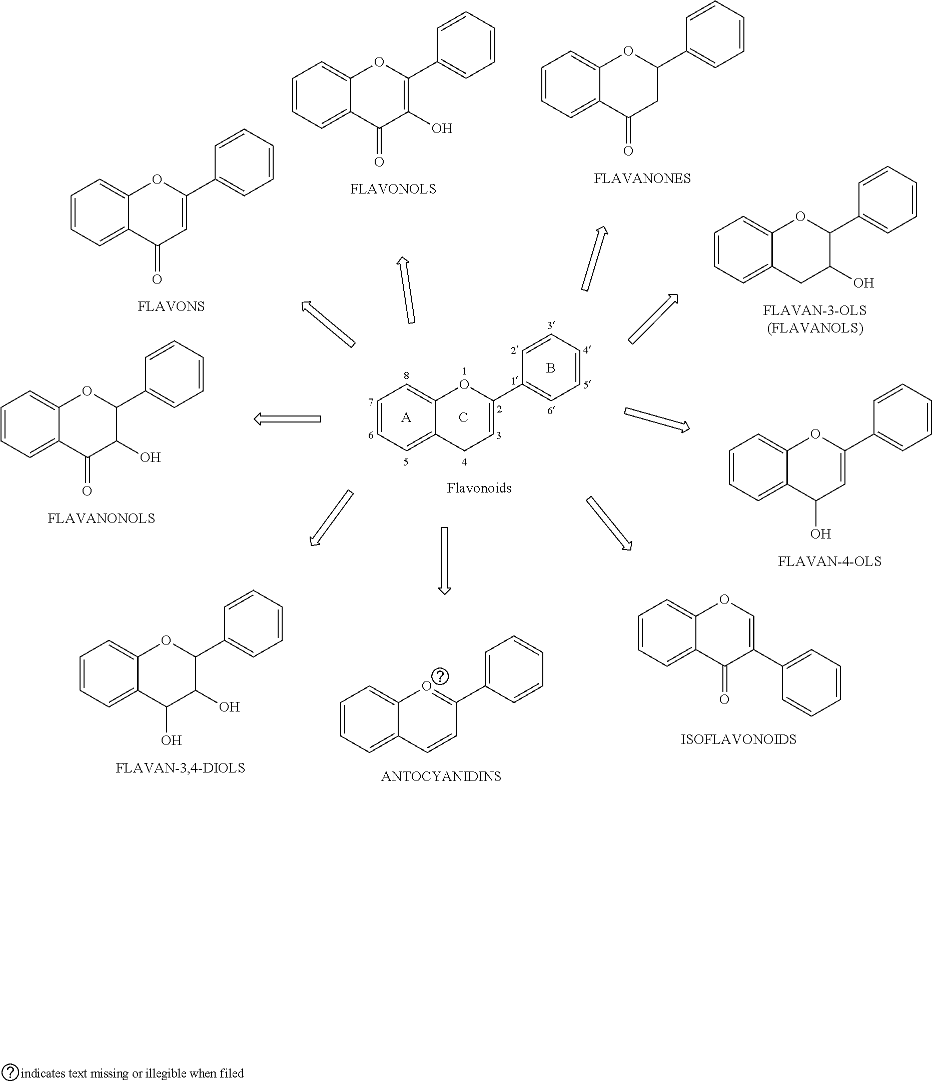

Preferred flavonoid compounds of Formula I include:

EGCG (CAS #989-51-5), EC (CAS #490-46-0), EGC (CAS #970-74-1) or ECG (CAS #1257-08-5)

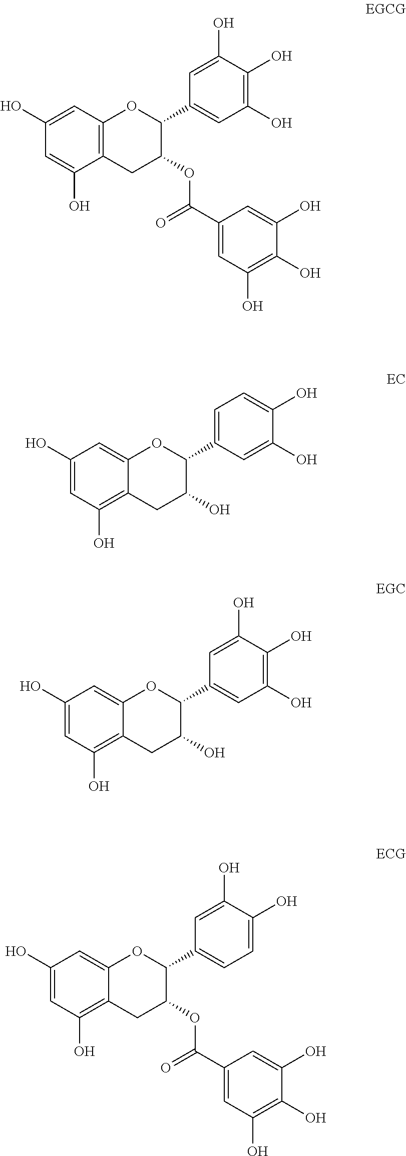 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Conjugate

The present invention provides a conjugate comprising: (a) a CNS-targeting ligand, (b) a hydrophilic polymer of polyethylene glycol (PEG), polylactic acid (PLA), polylactic-co-glycolic acid (PLGA), or dextran, and (c) a flavonoid of Formula I, wherein the PEG covalently binds to the flavonoid and the CNS-targeting ligand.

The conjugate targets CNS by the CNS-targeting ligand, and delivers active ingredients to the CNS tissue for treating CNS pathogenic conditions including but not limited to inflammation, neuron cell damage, pathogenic aggregates accumulation in CNS system.

The CNS-targeting ligand is covalently linked to PEG, PLA, PLGA, or dextran, either through its —COOH groups or its —NH₂ groups by a standard chemistry known to a person skilled in the art. The molecular weight of the hydrophilic polymer in the conjugate is in general 1K-100K, preferably 3K-80K, and more preferably 5K-40K.

The flavonoid in the conjugate has a general formula (I), and is preferably EGCG, EC, EGC, or ECG. In one embodiment, the flavonoid is epigallocatechin gallate (EGCG).

In one embodiment, PEG contains an aldehyde group which is conjugated to the 5, 6, 7, or 8 position (preferably 6 or 8 position) of the A ring of the flavonoid compound.

In another embodiment, PEG contains a thiol group which is conjugated to R₁ or R₂ of the B-ring of a flavonoid (when R₁ or R₂ is —OH).

In one embodiment, the conjugate comprises PEG-EGCG, which is PEG linked to one or two molecules of EGCG; which can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thiol group with the R₁ or R₂ of Formula I, wherein, R₁ or R₂ is a phenyl group. See WO2015/171079.

In another embodiment, the conjugate comprises PEG-EC, PEG-EGC, or PEG-ECG, and the conjugate can be prepared by conjugating aldehyde-terminated PEG to EC, EGC, or ECG by attachment of the PEG via reaction of the free aldehyde group with the 5, 6, 7, or 8 position (preferably 6 or 8 position) of Formula I.

HOOC-PEG-CHO and HO-PEG-CHO are commonly available. In one embodiment, HOOC-PEG-CHO is conjugated to EGCG, EC, EGC, or ECG according to WO2006/124000 and WO2009/054813. HOOC-PEG-flavonoid has COOH group to react with the N terminal of a CNS-targeting peptide. In general, a CNS-targeting peptide is incubated with HOOC-PEG-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CNS-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In another embodiment, HO-PEG-CHO is conjugated to EGCG, EC, EGC, or ECG according to WO2006/124000 and WO2009/054813. HO-PEG-flavonoid has OH group to react with the C terminal of a CNS-targeting peptide. In general, a peptide is incubated with HO-PEG-EGCG, and N, N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the CNS-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH on PEG, because the primary OH group on PEG is more reactive than the tertiary OH in the aromatic ring of flavonoid.

HOOC-PLA-CHO, HOOC-PLGA-CHO, and HO-Dextran-CHO are commercially available.

In one embodiment, HOOC-PLA-CHO is conjugated to EGCG, EC, EGC, or ECG according to WO2006/124000 and WO2009/054813. HOOC-PLA-flavonoid has COOH group to react with the N terminal of a CNS-targeting peptide. In general, a CNS-targeting peptide is incubated with HOOC-PLA-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CNS-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HOOC-PLGA-CHO is conjugated to EGCG, EC, EGC, or ECG according to WO2006/124000 and WO2009/054813. HOOC-PLGA-flavonoid has COOH group to react with the N terminal of a CNS-targeting peptide. In general, a CNS-targeting peptide is incubated with HOOC-PLGA-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CNS-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HO-Dextran-CHO is conjugated to EGCG, EC, EGC, or ECG according to WO2006/124000 and WO2009/054813. HO-Dextran-flavonoid has OH group to react with the C terminal of a CNS-targeting peptide. In general, a peptide is incubated with HO-Dextran-EGCG, and N, N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the CNS-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH in the CH₂OH terminal of dextran, because this is the only primary OH group in dextran, which is more reactive than other secondary OH in dextran and tertiary OH in the aromatic ring of flavonoid.

The CNS-targeting ligand in the present invention is a ligand selected to target receptors on a CNS cell (neuron cells) surface or CNS environment. The CNS-targeting ligands of the present invention, for example, target the following receptors on neuron cell or CNS environment:

(i) Receptors on neuron cells: This group includes but not limited to glutamatergic neuron receptors, GABAergic neuron receptors, dopaminergic neuron receptors, serotonergic neuron receptors, cholinergic neuron receptors, nicotinic receptors, muscarinic receptors, dopamine receptors, adenosine receptors, glutamate receptors, GABA receptors, AMPA receptors, NMDA receptors, TrkB receptors, CB1 receptors, scavenger receptors, synaptophysin, PSD95, VGLUT1, VGLUT2, NMDAR1, NMDAR28, GAT1, DAT, SERT, Pet1, and VAChT.

(ii) Receptors present in CNS environment: This group includes but not limited to oligodendrocyte receptors, oligodendrocyte precursor cell receptors, intermediate progenitor receptors, neuroepithelial cell receptors, Schwann cell receptors, radial glia receptors, astrocyte receptors, microglia receptors, pericyte receptors, B cell receptors, T cell receptors, RAGE receptor, Fc receptor, toll like receptors, TfR, IR, LDLR, Dhh, P75NTR, NCAM, E-cadherin, N-cadherin, PDGFRA, NG2, MOG, TN-C-glycoprotein, α(2-3)-sialoglycoprotein receptor, Notch, E-cadherin, S100, MBP, MPZ, EAAT1, TMEM119, CD11b, CD45, CX3CR1, F4/80, CD68, and CD40.

In one embodiment, the CNS-targeting ligand is TfR peptide having the amino acid sequence of THRPPMWSPVWP (SEQ ID NO: 1), which targets transferrin receptor on neuron cells or microenvironment in CNS.

In one embodiment, the CNS-targeting ligand is Tet1 peptide having the amino acid sequence of HLNILSTLWKYRC (SEQ ID NO: 2), which targets GT1b receptor on neuron cells or microenvironment in CNS.

In one embodiment, the CNS-targeting ligand is TC13 (TGN) peptide having the amino acid sequence of TGNYKALHPHNGC (SEQ ID NO: 3), which targets on neuron cells or microenvironment in CNS.

In one embodiment, the CNS-targeting ligand is Apamin peptide having the amino acid sequence of CNCKAPETALCARRCQQH (SEQ ID NO: 4), which targets apamin receptor on neuron cells or microenvironment in CNS.

In one embodiment, the CNS-targeting ligand is Regulon polypeptides having the amino acid sequence of PTVIHGKREVTLHL (SEQ ID NO: 5), which targets low density lipoprotein (LDL) receptor on neuron cells or microenvironment in CNS.

In one embodiment, the CNS-targeting ligand is RAP peptide having the amino acid sequence of ELKHFEAKIEKHNHYQKQLE (SEQ ID NO: 6), which targets LDL receptor on neuron cells or microenvironment in CNS.

In one embodiment, the CNS-targeting ligand is Angiopep-2 peptide having the amino acid sequence of TFFYGGSRGKRNNFKTEEY (SEQ ID NO: 7), which targets LDL receptor on neuron cells or microenvironment in CNS.

In one embodiment, the CNS-targeting ligand is TAT peptide having the amino acid sequence of GGGGYGRKKRRQRRR (SEQ ID NO: 8), which targets on neuron cells or microenvironment in CNS.

In one embodiment, the CNS-targeting ligand is SynB1 peptide having the amino acid sequence of RGGRLSYSRRRFSTSTGR (SEQ ID NO: 9), which targets on neuron cells or microenvironment in CNS.

In one embodiment, the CNS-targeting ligand is Leptin 30 peptide having the amino acid sequence of YQQVLTSLPSQNVLQIANDLENLRDLLHLLC (SEQ ID NO: 10), which targets leptin receptor on neuron cells or microenvironment in CNS.

In one embodiment, the CNS-targeting ligand is LNP peptide having the amino acid sequence of KKRTLRKNDRKKRC (SEQ ID NO: 11), which targets Caveolae-mediated endocytosis and macropinocytosis on neuron cells or microenvironment in CNS.

In one embodiment, the CNS-targeting ligand is ApoB peptide having the amino acid sequence of SSVIDALQYKLEGTTRLTRKRGLKLATALSLSNKFVEGS (SEQ ID NO: 12), which targets LRP2 receptor on neuron cells or microenvironment in CNS.

In one embodiment, the CNS-targeting ligand is RVG-29 peptide having the amino acid sequence of YTIWMPENPRPGTPCDIFTNSRGKRASNG (SEQ ID NO: 13), which targets nAChR receptor on neuron cells or microenvironment in CNS.

In one embodiment, the CNS-targeting ligand is T7 peptide having the amino acid sequence of HAIYPRH (SEQ ID NO: 14), which targets transferrin receptor on neuron cells or microenvironment in CNS.

In one embodiment, the CNS-targeting ligand is GSH (glutathione) peptide having the amino acid sequence of ECG, which targets on neuron cells or microenvironment in CNS.

In one embodiment, the CNS-targeting ligand is CRT peptide having the amino acid sequence of CRTIGPSVC (SEQ ID NO: 15), which targets transferrin receptor on neuron cells or microenvironment in CNS.

In one embodiment, the CNS-targeting ligand is CAQK peptide having the amino acid sequence of CAQK (SEQ ID NO: 16), which targets Proteoglycan complex on neuron cells or microenvironment in CNS.

In one embodiment, the CNS-targeting ligand is TACL05 peptide having the amino acid sequence of SACPSHLTKMCGGG (SEQ ID NO: 17), which targets neuron cells or microenvironment in CNS.

In one embodiment, the CNS-targeting ligand is adenosine, which targets adenosine A1 receptors.

In one embodiment, the CNS-targeting ligand is 5′-N-ethylcarboxamidoadenosine (NECA), which targets adenosine A2A receptors.

In one embodiment, the CNS-targeting ligand is glutamate, which targets glutamate receptors.

In one embodiment, the CNS-targeting ligand is γ-aminobutyric acid (GABA), which targets 5-HT4 receptor.

### Nanoparticle Composition

The term “MINC” (Multi-pathway Immune-modulating Nanocomplex Combination therapy) is a platform technology. The present invention provides a nanoparticle micelle (MINC) composition having an outer shell comprising one or more CNS-targeting conjugates of the present invention, optionally an inner shell comprising one or more flavonoid oligomers, and optionally a drug encapsulated within the shells.

In one embodiment, the micelle composition comprises both the outer shell and the inner shell as described above; the composition optionally has a drug encapsulated with the shells.

In one embodiment, the micelle composition comprises the outer shell as described above and does not have an inner shell; the composition optionally has a drug encapsulated with the shell.

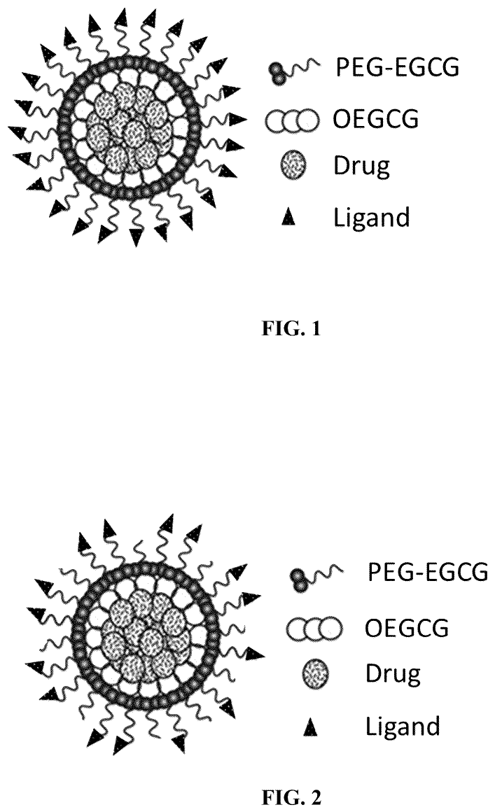

In one embodiment, the micelle composition comprises CNS-targeting ligand-polymer-flavonoid conjugate in an outer shell and oligomeric flavonoid in an inner shell, wherein the flavonoid in the outer shell and the flavonoid in the inner shell are independently EGCG, EC, EGC, or ECG, and the polymer is PEG, PLA, PLGA, or dextran. A preferred polymer is PEG. A preferred flavonoid is EGCG. FIG. 1 shows a preferred micelle composition. The CNS-targeting ligand allows the nanoparticle composition to specifically target the CNS tissues.

In one embodiment, the micelle outer shell further comprises a bare PEG-flavonoid conjugate such as PEG-EGCG, which does not have a CNS-targeting ligand linked to PEG-flavonoid. See FIG. 2. In such a micelle outer shell, the ratio of ligand-PEG-EGCG to ligand-PEG-EGCG plus PEG-EGCG is in general more than 10%, or more than 20%, or more than 30%, or more than 50%, and up to 100%. In one embodiment, the ratio of ligand-PEG-EGCG to ligand-PEG-EGCG plus PEG-EGCG is 10-90%, or 20-80%, or 40-60%.

The micelles optionally comprise a CNS-treating molecule (a drug or an agent) encapsulated within the micelle (MINC-agent)

In one embodiment, the MINC-agent composition comprises three active ingredients, which are complementary in function to tackle both immune response and signaling pathways by its backbone components (PEG-flavonoid/OEGCG), and additional signaling pathways by a selected drug molecule for treating CNS diseases. Each nanoparticle is a fixed-dose combination drug with the three active ingredients at fixed molar ratio.

The present invention delivers MINC-agent to targeted CNS tissues by active delivery of the micelles through a CNS-targeting ligand to brain with specific receptors.

The nanocomplex of the present invention contains the first two active ingredients, flavonoid such as OEGCG and PEG-flavonoid such as PEG-EGCG in the backbone of the micelle composition. They are derivatives of EGCG, which is a strong immune modulator and regulates a wide spectrum of disease signaling pathways. EGCG regulates both innate and adaptive immunity. However, the bioavailability of EGCG is low and EGCG is not stable. The present nanocomplex composition overcomes the bioavailability issue of EGCG by forming a nanocarrier to carry EGCG to a target site for treatment and overcomes the stability issue of EGCG by forming OEGCG and PEG-EGCG complex, which effectively enables EGCG as highly effective therapeutic agents.

The nanocomplex of the present invention optionally contains a third active ingredient, which is a drug molecule encapsulated in the nanoparticles for treating CNS diseases. In one embodiment, the CNS disease is Alzheimer's disease, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-β amyloid, anti-tau, anti-IL6R, anti-IL-1β, anti-CD38, anti-TREM2, GDNF, NRTN, PDGF-BB, CDNF, or BDNF.

In one embodiment, the CNS disease is Parkinson's disease, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-α-synuclein, anti-IL6R, anti-IL-1β, anti-CD38, anti-TREM2, GDNF, NRTN, PDGF-BB, CDNF, or BDNF.

In one embodiment, the CNS disease is Lewy body dementia, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-β amyloid or, anti-α-synuclein, anti-IL6R, anti-IL-1β, anti-CD38, anti-TREM2, GDNF, NRTN, PDGF-BB, CDNF, or BDNF.

In one embodiment, the CNS disease is brain tumor, and the drug is doxorubicin, disulfiram, celecoxib, temsirolimus, everolimus, vorinostat, cabozantinib, marizomib, fimepinostat, acetazolamide, metformin, vinblastine, cyclophosphamide, anti-HER2, anti-EGFR, anti-PD-1, anti-PD-L1, anti-PDGFRA, anti-VEGF, anti-VEGFR2, IL-2, IL-4, IL-12, IFN-α, IFN-β, IFN-γ, or TNF-α.

In one embodiment, the CNS disease is stroke, and the drug is MMP inhibitor, eNOS inhibitor, anti-TLR4, anti-HSP, anti-IL6, anti-IL-12, S100β, Fibronectin, MCP-1, MMP9, UCH-L1, BDNF, GDNF, NRTN, PDGF-BB, or CDNF.

In one embodiment, the CNS disease is Huntington's disease, and the drug is anti-CD3, anti-mHtt, anti-α-synuclein, anti-SEMA4D, anti-TNFα, Tetrabenazine, deutetrabenazine, valbenazine, bevantolol, pridopidine, branaplam, nilotinib, mitoconix, or azathioprine

In one embodiment, the CNS disease is multiple sclerosis, and the drug is anti-CD3, anti-CD4, anti-IL-17, anti-CD19, anti-CD20, anti-CD25, anti-CD52, anti-RGMA, anti-IL-12, anti-IL-23, anti-α4 integrin, anti-IL-2R, LINGO-1, or anti-NOGO-A.

In one embodiment, the CNS disease is amyotrophic lateral sclerosis (ALS), and the drug is anti-NOGO-A, PKC inhibitor, IGF-1, NOGO-A, GDNF, VEGF, anti-SOD1, SIR, GLT-1, anti-Ataxin2, anti-TDP43, anti-hnRNPs, CK-1 inhibitor, anti-FET or HDAC inhibitor, EPO, or IL-2.

In one embodiment, the CNS disease is acute spinal cord injury, and the drug is Extracellular domain of Nogo receptor, 5-HT1A receptor, FGF, GSK-3bβ inhibitor, anti-IN-1, TNF-α, IL-12, SDF-1α, SOD1, NEC-1, anti-P-selectin, or anti-CD11d.

In one embodiment, the CNS disease is encephalitis, and the drug is anti-FcRn, anti-IL-6, anti-CD20, anti-CD19, anti-CD38, anti-C5, or IL-2.

In one embodiment, the CNS disease is epilepsy or seizures, and the drug is mTOR inhibitor, PI3K inhibitor, GABA inhibitor, anti-Glu3B peptide antibody, anti-NR1 antibody, anti-CASPR2, or anti-LGI-1.

In one embodiment, the CNS disease is meningitis, and the drug is C1 inhibitor, anti-C5, anti-MASP-2, anti-PD-L1, anti-CTLA-4, or anti-PD-1.

In one embodiment, the CNS disease is motor neuron disease (MND), and the drug is Anti-SOD1, anti-TDP-43, anti-C90RF72, anti-Nogo-A, anti-MuSK, anti-IL-6R, anti-NRP-1, anti-Myostatin, anti-CD40L, anti-DR-6, anti-IFN-g, anti-GD1a, anti-CTGF, or anti-HMGB1.

The nanoparticle is stable in a hydrophilic environment, such as blood circulation, and dissociates in a hydrophobic environment, such as a CNS tissue.

### Process for Preparing the Nanoparticle Composition

The nanoparticle composition of the present invention can be prepared by a process comprises the steps of: (a) mixing a drug molecule with flavonoid oligomer (e.g., OEGCG) and the CNS-targeting ligand conjugate of the present invention in an aqueous solution; and (b) filtering the mixture through a membrane with a molecular weight cut-off of 8,000-300,000 daltons to remove small molecular weight molecules and retain large molecular weight molecules.

In one preferred embodiment, the process further comprises step (c), filtering the large molecular weight molecules through 0.2-0.3 μm membrane and collecting the filtrate.

In step (a), the drug molecule is dissolved in an aqueous solvent, such as phosphate-buffer saline, saline, water, bicarbonate buffer, oxyhemoglobin buffer, bis-tris alkane, Tris-HCl, HEPES, histidine buffer, NP-40, RIPA (radioimmunoprecipitation assay buffer), tricine, TES, TAPS, TAPSO, Bicine, MOPS, PIPES, cacodylate, or MES. Preferred solvents are phosphate-buffer saline, saline, or water. The protein drug concentration is in general 0.01-50 mg/ml, preferred 0.05-10 mg/ml, and more preferred 0.1-5 mg/ml.

The flavonoid oligomer and the CNS-targeting ligand conjugate are dissolved in ketones, acetonitrile, alcohols, aldehydes, ethers, acetates, sulfoxides, benzenes, organic acids, amides, aqueous buffers, and any combination thereof. Preferred solvents are alcohols, acetonitrile, sulfoxides, amides, and any combination thereof. For example, the OEGCG/EGCG and PEG-EGCG concentrations are in general independently 0.001-10 mg/ml, preferred 0.005-1 mg/ml, or 0.1-5 mg/ml.

It is important that OEGCG is in molar excess of the drug agent. In general, the molar ratio of the EGCG in OEGCG to the drug molecule is between 1-500 to 1, 2-500 to 1, 3-500 to 1, or 5-500 to 1, preferably 3-100 to 1, 5-100 to 1, or 10-50 to 1. The molar ratio is calculated by the number of moles of monomer EGCG in OEGCG to the number of moles of the drug molecule. The molar excess of EGCG ensures most or all drug agents are encapsulated by the OEGCG molecules. Unencapsulated drug agents, which would not be selectively distributed to target tissue and would cause lower efficacy and safety issues, are avoided by controlling the molar ratio of OEGCG to protein in the present process.

In step (b), the above mixture is filtered through a membrane with a molecular weight cut-off between 8,000-300,000 daltons, preferably between 8,000-200,000 daltons, 8,000-150,000 daltons, or 8,000-12,000 daltons, to remove small molecular weight molecules and retain large molecular weight molecules. The ultrafiltration membrane material is selected from the group consisting of cellulose (and its derivatives), polyethersulfone (PES), polytetrafluoroethylene (PTFE), nylon, polyvinylidene fluoride or polyvinylidene difluoride (PVDF), and polypropylene (PP); preferably cellulose (and its derivatives), PTFE, and PVDF.

The mixture is optionally diluted in an aqueous solvent such as those described above in step (a) before ultrafiltration.

The ultrafiltration step (b) removes unwanted impurities of small molecular weight, such as unreacted OEGCG or EGCG, or reaction by-products. These impurities may reduce drug efficacy and safety. The excess of unreacted OEGCG or EGCG may also lead to aggregation of the individual nanoparticles to about 1000 nm size particles, which would reduce efficacy and cause potential toxicity.

In step (c), the retained large molecular weight molecules are filtered through a membrane having a pore size of about 0.2-0.3 μm, such as 0.22 μm, and the filtrate is collected. This is to remove unwanted impurities of large molecular sizes, such as mega-aggregates. These aggregates may be excreted from entering tissues due to its mega size. These aggregates reduce overall efficacy/safety and have a higher chance of inducing immunogenicity to the patients. Large size nanoparticles are also easier to be taken up by RES in the liver, lungs, and more undesired organs.

The membrane material of step (c) is selected from the group consisting of cellulose (and its derivatives), PES, PTFE, nylon, PVDF, and PP; preferably cellulose (and its derivatives), PES, and PP.

In one embodiment, the steps (b) and (c) are repeated at least one time, for example, repeated 1, 2, 3, or 4 times before step (d), to effectively remove unwanted small molecule impurities and large aggregates.

After step (c), the filtrate is stored at 2-8° C., and is stable for at least 100 days.

The present process optionally further comprises a lyophilization step (d) after step (c) to provide a long-term stability of the nanoparticle composition.

### Pharmaceutical Compositions

The present invention provides pharmaceutical compositions comprising the nanoparticle composition of the present invention and optionally one or more pharmaceutically acceptable carriers. The nanoparticle composition in a pharmaceutical composition in general is about 1-90%, preferably 20-90%, or 30-80% for a tablet, powder, or parenteral formulation. The nanoparticle composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

### Method of Treatment

The present invention is directed to a method of treating CNS diseases, comprising the step of administering an effective amount of the nanoparticle composition of the present invention to a subject in need thereof. Suitable CNS to be treated by the present invention include but not limited to Alzheimer's disease, Parkinson's disease, Huntington's disease, migraine, multiple sclerosis, autism, cerebral palsy, epilepsy (seizures), amyotrophic lateral sclerosis, spinal cord injury.

“An effective amount,” as used in this application, is the amount effective to treat a disease by ameliorating the pathological condition or reducing the symptoms of the disease.

Dosing for a ligand-polymer-flavonoid, e.g., ligand-polymer-EGGC, for injection, is in general 0.01 to 100 mg/kg (total weight of the polymer-flavonoid/subject body weight), or 0.001 to 1000 mg/kg.

In one embodiment, the method comprises the step of administering to a subject in need thereof an effective amount of MINC having a shell formed by one or more ligand-hydrophilic polymer-flavonoid conjugates and optionally with a bare polymer-flavonoid conjugate, with or without flavonoid oligomers, optionally having an agent encapsulated within the shell.

In one embodiment, the shell is formed by ligand-PEG-EGCG, optionally with PEG-EGCG.

In one embodiment, the shell is formed by ligand-PEG-EGCG and OEGCG, and optionally PEG-EGCG.

The ligand-polymer-flavonoid conjugate of the present invention is capable of crossing the Blood-Brain Barrier (BBB) from the circulating blood vessel to the brain. The ligand-polymer-flavonoid conjugate of the present invention is capable of targeting to CNS lesions after BBB penetration. The ligand polymer-flavonoid conjugate of the present invention additionally has neuron cell repair or regeneration activity, and has immune and disease-modulating functions for treating CNS disorders.

The flavonoid oligomer used in the present method is capable of crossing the BBB from the circulating blood vessel to the brain, and has immune and disease-modulating functions for treating CNS disorders. The flavonoid oligomer of the present invention additionally has neuron cell repair or regeneration activity for treating CNS disorders.

Dosing of the MINC-agent is based on the known dosage of the agent for treating a particular disease and the subject condition. The dosage can be a food drug administration (FDA) approved dosage or a dosage used in clinical trial.

In MINC-agent, the total weight of ligand-PEG-EGCG and PEG-EGCG if present, is close to the encapsulated drug agent. The weight of OEGCG, if present, varies. In general, the dosage of ligand-PEG-EGCG and PEG-EGCG if present, combined with OEGCG is between 0.01 to 1000 mg/kg.

The concentration for the encapsulated drug agents can be as low as 0.01 mg/kg (e. g., for cytokine drugs, rhBDNF) and as high as 100 mg/kg (for antibody drugs, e.g., anti-α synuclein antibody is at this level).

For treating Alzheimer's disease, anti-β amyloid (Aducanumab, Solanezumab, Crenezumab, Gantenerumab, Donanemab or Lecanemab) is given at 0.01-100 mg/kg or 0.01-1000 mg/kg IV every one to four weeks. The effective dose of MINC-anti-β amyloid in the same dose range can be used for treating Alzheimer's disease.

For treating Parkinson's disease, anti-α synuclein (Prasinezumab or Cinpanemab) is given at 0.01-100 mg/kg or 0.01-1000 mg/kg IV every one to four weeks. The effective dose of MINC-anti-α synuclein in the same dose range can be used for treating Parkinson's disease.

For treating Huntington's disease, anti-mHtt (C6/17) is given at 0.01-100 mg/kg or 0.01-1000 mg/kg IV every one to four weeks. The effective dose of MINC-anti-mHtt in the same dose range can be used for treating Huntington's disease.

For treating migraine, anti-CGRP (Eptinezumab, Fremanezumab, Galcanezumab, Erenumab) is given at 0.01-100 mg/kg or 0.01-1000 mg/kg IV every one to four weeks. The effective dose of MINC-anti-CGRP in the same dose range can be used for treating migraine.

For treating multiple sclerosis, anti-CD20 (Ocrelizumab, Rituximab, Ofatumumab or Ublituximab) is given 0.01-100 mg/kg or 0.01-1000 mg/kg IV once to four times per year. The effective dose of MINC-anti-CD20 in the same dose range can be used for treating multiple sclerosis.

The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

### Example 1: TfR Peptide Conjugation to HOOC-PEG-EGCG

### Materials

TfR Peptide was purchased from Hangzhou Xinbosi Biomedical

HOOC-PEG-CHO was purchased from NBC chemical

### Method

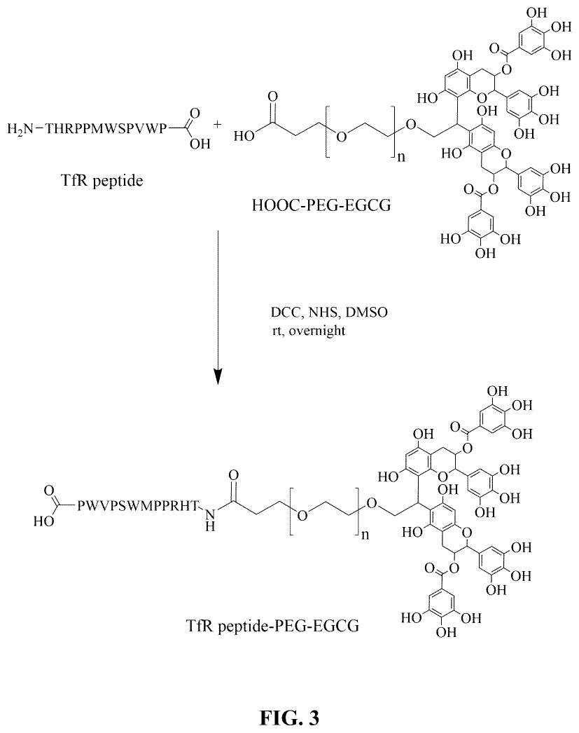

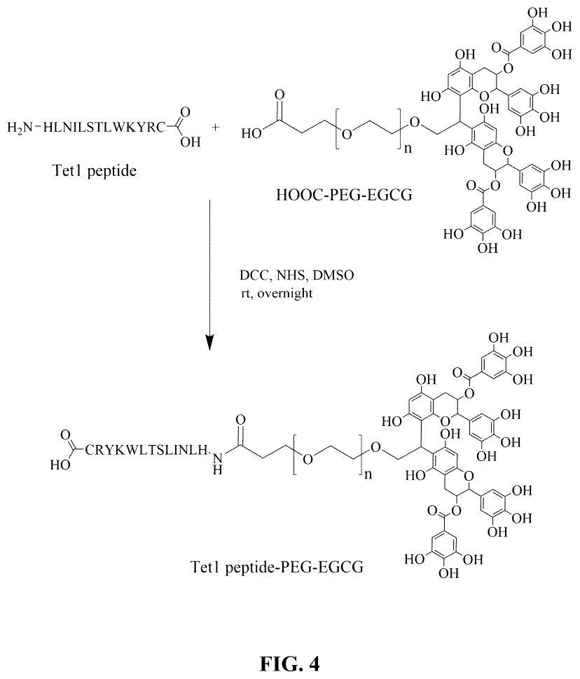

HOOC-PEG-CHO was conjugated to EGCG according to WO2006/124000 and WO2009/054813; TfR peptide was conjugated to HOOC-PEG-EGCG via the conjugation between COOH group on PEG and NH₂ group on TfR to form TfR-PEG-EGCG (N′ linked) (FIG. 3).

Specifically, 1-1000 mg TfR was PEGylated by incubation with 1-1000 mg HOOC-PEG-EGCG, 1-1000 mg N, N′-dicyclohexylcarbodiimide (DCC), and 1-1000 mg N-hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of TfR-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after TfR conjugation, a new peak with retention time at 6.46 min was present. The HPLC results indicate a successful conjugation of TfR-PEG-EGCG.

### Example 2: Tet1 Peptide Conjugation to HOOC-PEG-EGCG

### Materials

Tet1 Peptide was purchased from Hangzhou Xinbosi Biomedical

HOOC-PEG-CHO was purchased from NBC chemical

### Method

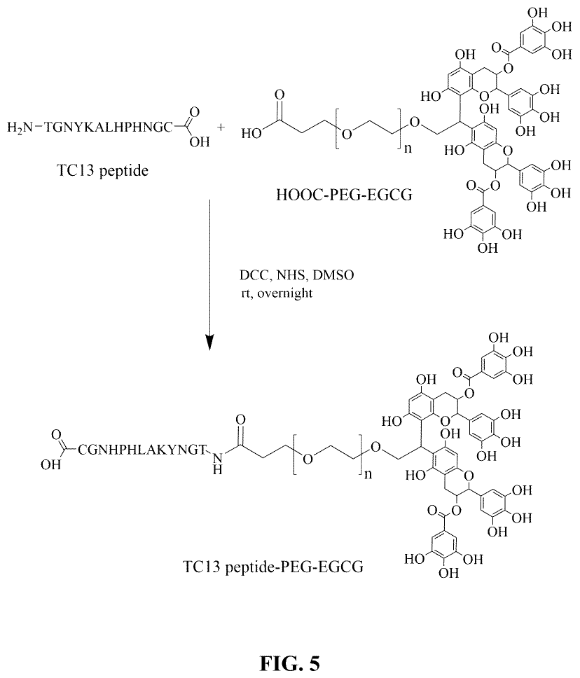

HOOC-PEG-CHO was conjugated to EGCG according to WO2006/124000 and WO2009/054813; Tet1 peptide was conjugated to HOOC-PEG-EGCG via the conjugation between COOH group on PEG and NH₂ group on Tet1 to form Tet1-PEG-EGCG (N′ linked) (FIG. 4).

Specifically, 1-1000 mg Tet1 was PEGylated by incubation with 1-1000 mg HOOC-PEG-EGCG, 1-1000 mg N, N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of Tet1-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after Tet1 conjugation, a new peak with retention time at 7.54 min was present. The HPLC results indicate a successful conjugation of Tet1-PEG-EGCG.

### Example 3: TC13 Peptide Conjugation to HOOC-PEG-EGCG

### Materials

TC13 Peptide was purchased from Hangzhou Xinbosi Biomedical

HOOC-PEG-CHO was purchased from NBC chemical

### Method

HOOC-PEG-CHO was conjugated to EGCG according to WO2006/124000 and WO2009/054813; TC13 peptide was conjugated to HOOC-PEG-EGCG via the conjugation between COOH group on PEG and NH₂ group on TC13 to form TC13-PEG-EGCG (N′ linked) (FIG. 5).

Specifically, 1-1000 mg TC13 was PEGylated by incubation with 1-1000 mg HOOC-PEG-EGCG, 1-1000 mg N, N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of TC13-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after TC13 conjugation, a new peak with retention time at 6.54 min was present. The HPLC results indicate a successful conjugation of TC13-PEG-EGCG.

### Example 4: Preparing TfR-MINC-Doxorubicin, Tet1-MINC-Doxorubicin and TC13-MINC-Doxorubicin

### Materials

TfR-PEG-EGCG was prepared according to Example 1.

Tet1-PEG-EGCG was prepared according to Example 2.

TC13-PEG-EGCG was prepared according to Example 3.

Doxorubicin was purchased from Sigma-Aldrich.

### Method

TfR-MINC-doxorubicin nanoparticles, Tet1-MINC-doxorubicin nanoparticles and TC13-MINC-doxorubicin nanoparticles were prepared according to the following protocol:
1. Incubate 5-500 μg doxorubicin in 1 mL DMSO for 15 min to 1 hour.
2. Add 1-100 μg of OEGCG and 1-10,000 μg of TfR-PEG-EGCG, Tet1-PEG-EGCG or TC13-PEG-EGCG. Incubate the mixture at 25° C. for 3 hours.
3. Filter out the liquid with a 10K MWCO filter unit. Wash the filter 3 time with 0.9% NaCl
4. Lyophilize to dry powder.

### Results

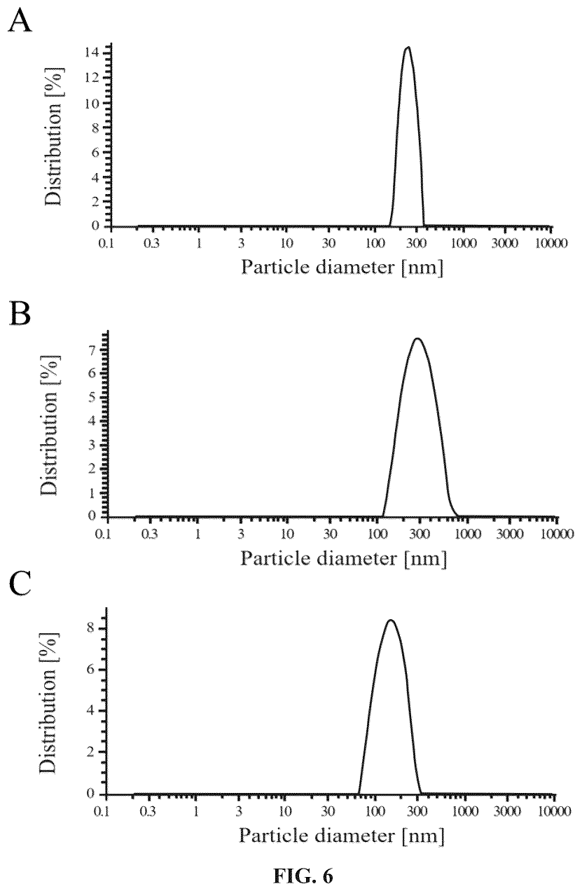

Nanoparticle size was measured by DLS (Anton Paar, Litesizer 500). FIG. 6 showed successful formulation of TfR-MINC-doxorubicin (A), Tet1-MINC-doxorubicin (B) and TC13-MINC-doxorubicin (C).

### Example 5: Different Ligand-MINC-Doxorubicin in Delivering Drugs into Brain Endothelial Cells

### Materials

TfR-MINC-doxorubicin, Tet1-MINC-doxorubicin, TC13-MINC-doxorubicin or MINC-doxorubicin were formulated according to Example 4.

### Method

Brain endothelial cell line bEnd.3 was seeded at 8×103 cells/well in 96 well plate with and incubated overnight. On the second day, cells were treated with MINC-doxorubicin, TfR-MINC-doxorubicin, Tet1-MINC-doxorubicin or TC13-MINC-doxorubicin at 2.5 M for 2 hours (n=2). After the treatment, the fluorescence intensity was measured by Molecular Devices Gemini XPS Fluorescent Microplate Reader to assess the delivery efficiency. Data are shown as means±SD, and statistically analyzed by GraphPad Prism 7. The statistical significance was calculated by one-way ANOVA and differences were considered to be significant at *: p<0.05, **: p<0.01; ***: p<0.001.

### Result

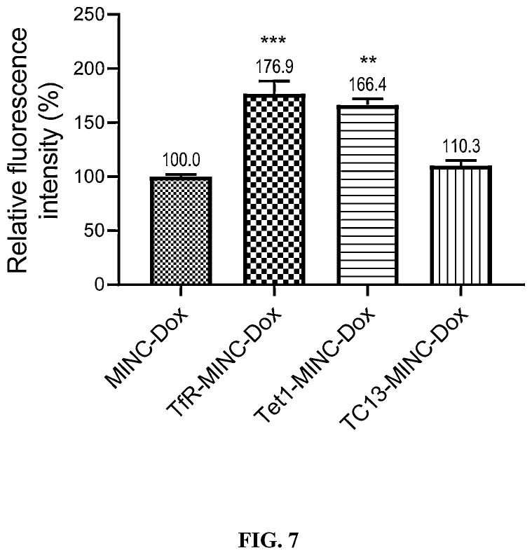

Doxorubicin is a red fluorescent compound, its delivery into cells was observed using fluorescence microscope. The higher fluorescence intensity means more doxorubicin was delivered into the cells. In FIG. 7, compared to MINC-doxorubicin, the fluorescent signal of TfR-MINC-doxorubicin and Tet1-MINC-doxorubicin were significantly stronger in the bEnd.3 cells. We also observed a trend of higher fluorescence signal in TC13-MINC-doxorubicin treatment cells. These results demonstrate that the CNS-targeting peptides increased specific drug delivery into brain endothelial cells.

### Example 6: TfR Peptide Conjugation to HO-PEG-EGCG (Prophetic Example)

### Objectives

This experiment is intended to demonstrate the conjugation of TfR peptide to HO-PEG-EGCG. HPLC is used to detect the formation of new product (TfR-PEG-EGCG) with different retention time from HO-PEG-EGCG. NMR can be used to confirm the structure.

### Materials

TfR Peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

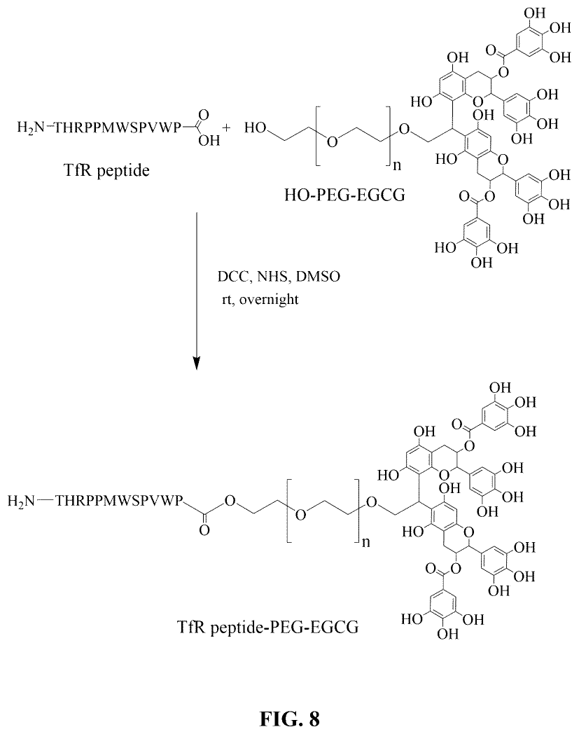

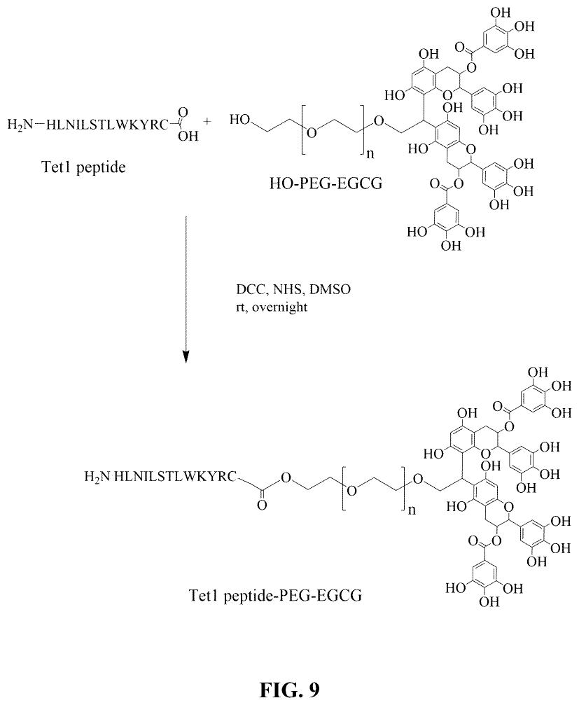

TfR peptide is conjugated to HO-PEG-EGCG via the conjugation between OH group on PEG and COOH group on TfR to form TfR-PEG-EGCG (C′ linked). See FIG. 8.

Specifically, 1-1000 mg TfR is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 7: Tet1 Peptide Conjugation to HO-PEG-EGCG (Prophetic Example)

### Objectives

This experiment is intended to demonstrate the conjugation of Tet1 peptide to HO-PEG-EGCG. HPLC is used to detect the formation of new product (Tet1-PEG-EGCG) with different retention time from HO-PEG-EGCG. NMR can be used to confirm the structure.

### Materials

Tet1 Peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

Tet1 peptide is conjugated to HO-PEG-EGCG via the conjugation between OH group on PEG and COOH group on Tet1 to form Tet1-PEG-EGCG (C′ linked). See FIG. 9.

Specifically, 1-1000 mg Tet1 is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 8: TC13 Peptide Conjugation to HO-PEG-EGCG (Prophetic Example)

### Objectives

This experiment is intended to demonstrate the conjugation of TC13 peptide to HO-PEG-EGCG. HPLC is used to detect the formation of new product (TC13-PEG-EGCG) with different retention time from HO-PEG-EGCG. NMR can be used to confirm the structure.

### Materials

TC13 Peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

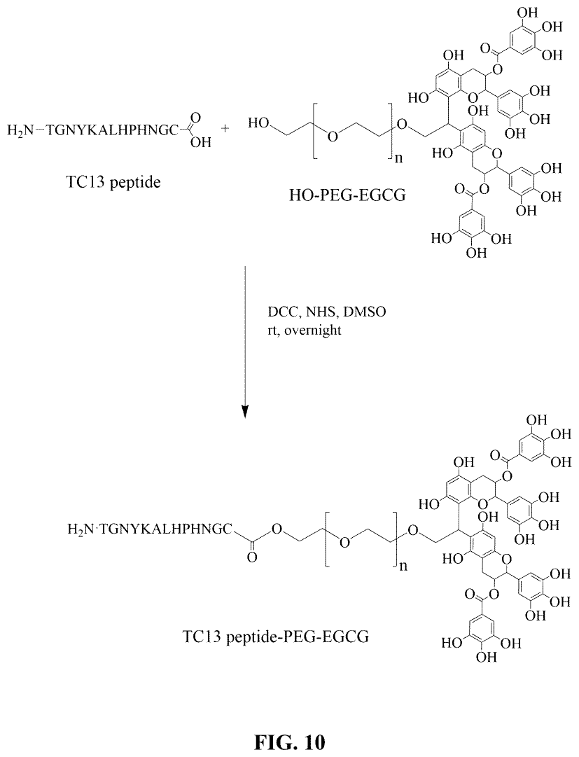

TC13 peptide is conjugated to HO-PEG-EGCG via the conjugation between OH group on PEG and COOH group on TC13 to form TC13-PEG-EGCG (C′ linked). See FIG. 10.

Specifically, 1-1000 mg TC13 is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 9: Adenosine Conjugation to HOOC-PEG-EGCG (Prophetic Example)

### Objectives

This experiment is intended to demonstrate conjugation of adenosine to HOOC-PEG-EGCG. HPLC is used to detect the formation of new product (adenosine-PEG-EGCG) with different retention time from HO-PEG-EGCG. NMR can be used to confirm the structure.

### Materials

Adenosine is purchased from sigma aldrich

HOOC-PEG-CHO is purchased from NBC chemical

### Method

HOOC-PEG-CHO is conjugated to EGCG according to WO2006/124000 and WO2009/054813.

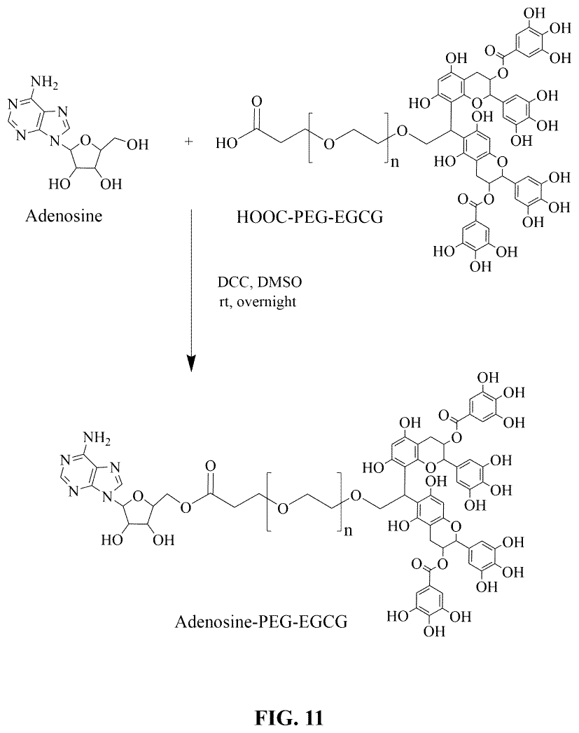

Adenosine is conjugated to HOOC-PEG-EGCG via the conjugation between COOH group on PEG and COOH group on adenosine to form adenosine-PEG-EGCG. See FIG. 11

Specifically, 1-1000 mg adenosine is PEGylated by incubation with 1-1000 mg HOOC-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 10: TfR Peptide Conjugation to HOOC-PLA-EGCG (Prophetic Example)

### Objectives

This experiment is intended to demonstrate conjugation of TfR peptide to HOOC-PLA-EGCG. HPLC is used to detect the formation of new product (TfR-PLA-EGCG) with different retention time from HOOC-PLA-EGCG. NMR can be used to confirm the structure.

### Materials

TfR Peptide is purchased from Hangzhou Xinbosi Biomedical.

HOOC-PLA-CHO is purchased from Merck (Sigma-Aldrich).

### Method

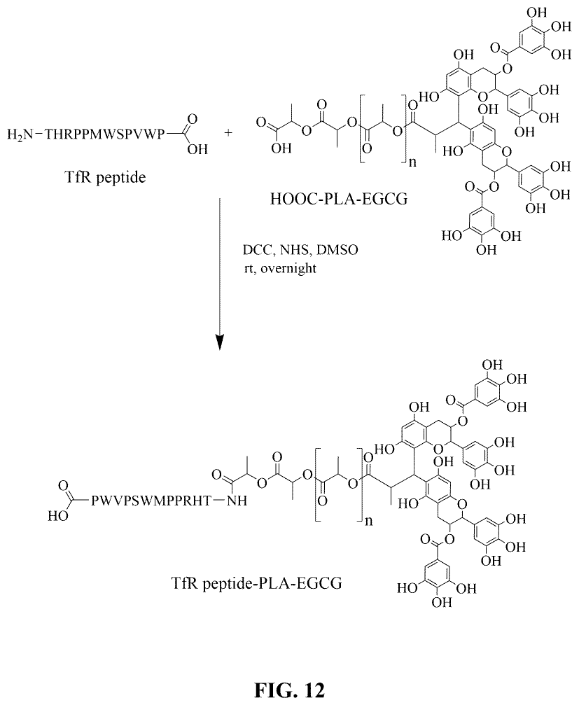

HOOC-PLA-CHO is conjugated to EGCG according to WO2006/124000 and WO2009/054813; TfR peptide is conjugated to HOOC-PLA-EGCG via the conjugation between COOH group on PLA and NH₂ group on TfR to form TfR-PLA-EGCG (N′ linked). See FIG. 12.

Specifically, 1-1000 mg TfR is incubated with 1-1000 mg HOOC-PLA-EGCG, 1-1000 mg N, N′-dicyclohexylcarbodiimide (DCC), and 1-1000 mg N-hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 11: TfR Peptide Conjugation to HOOC-PLGA-EGCG (Prophetic Example)

### Objectives

This experiment is intended to demonstrate conjugation of TfR peptide to HOOC-PLGA-EGCG. HPLC is used to detect the formation of new product (TfR-PLGA-EGCG) with different retention time from HOOC-PLGA-EGCG. NMR can be used to confirm the structure.

### Materials

TfR Peptide is purchased from Hangzhou Xinbosi Biomedical.

HOOC-PLGA-CHO is purchased from Merck (Sigma-Aldrich).

### Method

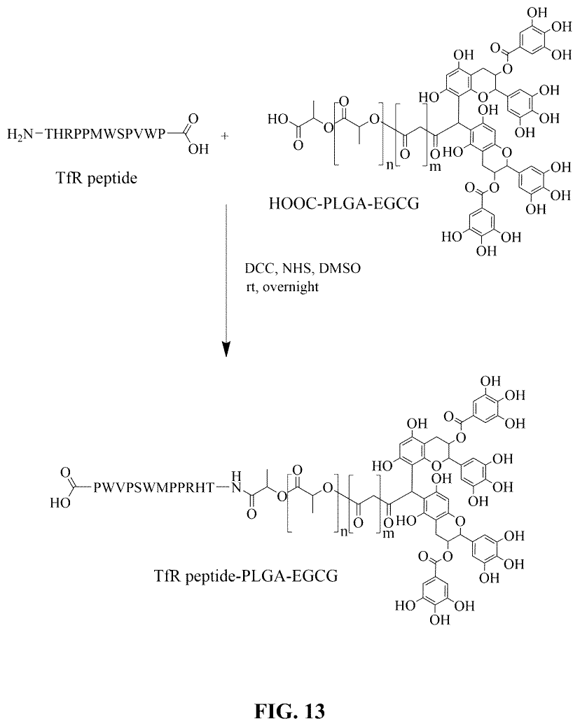

HOOC-PLGA-CHO is conjugated to EGCG according to WO2006/124000 and WO2009/054813; TfR peptide is conjugated to HOOC-PLGA-EGCG via the conjugation between COOH group on PLGA and NH₂ group on TfR to form TfR-PLGA-EGCG (N′ linked). See FIG. 13.

Specifically, 1-1000 mg TfR is incubated with 1-1000 mg HOOC-PLGA-EGCG, 1-1000 mg N, N′-dicyclohexylcarbodiimide (DCC), and 1-1000 mg N-hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 12: TfR Peptide Conjugation to HO-Dextran-EGCG (Prophetic Example)

### Objectives

This experiment is intended to demonstrate the conjugation of TfR to HO-Dextran-EGCG. HPLC is used to detect the formation of new product (TfR-Dextran-EGCG) with different retention time from HO-Dextran-EGCG. NMR can be used to confirm the structure.

### Materials

TfR peptide is purchased from Hangzhou Xinbosi Biomedical.

HO-Dextran-CHO is purchased from Merck (Sigma-Aldrich).

### Method

HO-Dextran-CHO is conjugated to EGCG according to WO2006/124000 and WO2009/054813 to prepare HO-Dextran-EGCG.

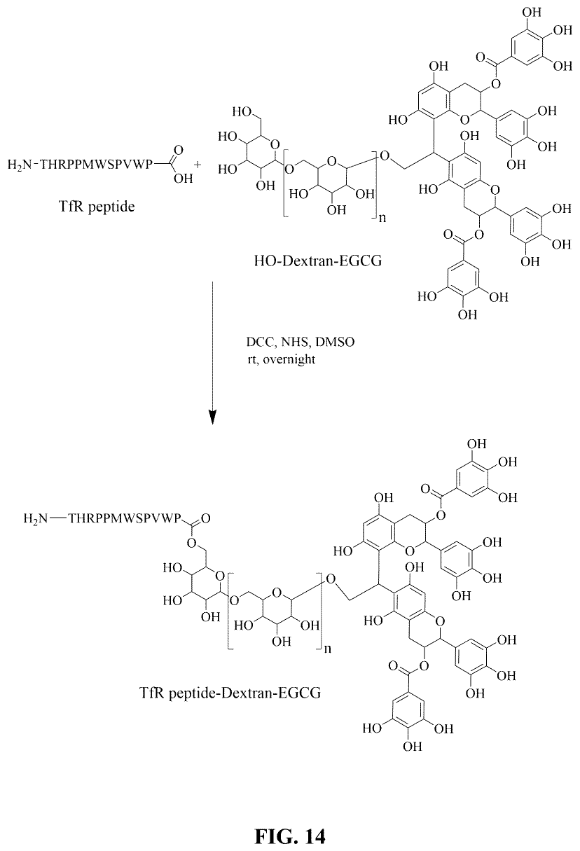

TfR is conjugated to HO-Dextran-EGCG via the conjugation between OH group on Dextran and COOH group on TfR to form TfR-Dextran-EGCG (C′ linked). See FIG. 14.

Specifically, 1-1000 mg TfR is incubated with 1-1000 mg HO-Dextran-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 13: Formulation of Adenosine-MINC-Doxorubicin (Prophetic Example)

### Objectives

This experiment is intended to demonstrate the formulation of adenosine-MINC-doxorubicin. DLS is used to measure the size of nanoparticle.

### Materials

Adenosin-PEG-EGCG is formulated according to Example 9.

Doxorubicin is purchased from Sigma-Aldrich or other suppliers.

### Method

Adenosinee-MINC-doxorubicin Nanoparticles are prepared according to the following protocol:
1. Incubate 5-500 μg doxorubicin in 1 mL DMSO for 15 min to 1 hour.
2. Add 1-100 μg of OEGCG and 1-10,000 μg of adenosinee-PEG-EGCG. Incubate the mixture at 25° C. for 3 hours.
3. Filter out the liquid with a 10K MWCO filter unit. Wash the filter 3 time with 0.9% NaCl
4. Lyophilize to dry powder.

### Example 14: Efficacy Study of TfR-MINC-Anti-CD3, Tet1-MINC-Anti-CD3, TC13-MINC-Anti-CD3 and Adenosine-MINC-Anti-CD3 in an Alzheimer's Disease Mouse Model (Prophetic Example)

### Objective

This experiment is intended to demonstrate the therapeutic efficacy of TfR, Tet1, TC13 peptide and adenosine conjugation. Histologic anti-Aβ staining is used to detect brain Aβ content. The behavior studies are used to evaluate the spatial working memory and exploratory activity.

### Materials

TfR-MINC-anti-CD3 nanoparticles, Tet1-MINC-anti-CD3 nanoparticles, TC13-MINC-anti-CD3 nanoparticles, adenosine-MINC-anti-CD3 and MINC-anti-CD3 nanoparticles are prepared using the method taught in Example 4 by replacing doxorubicin with anti-CD3.

Anti-CD3 antibody is purchased from Biolegend.

### Method

Tg APPsw (line 2576) mice, APP/PS1 mice or Wistar rats are used. The mice or rats are divided to treat with vehicle (PBS or saline as no treatment control), 5-125 g anti-CD3/mouse of MINC-anti-CD3, TfR-MINC-anti-CD3 nanoparticles, Tet1-MINC-anti-CD3 nanoparticles, TC13-MINC-anti-CD3 or adenosine-MINC-anti-CD3 via iv injection once per week for 4-8 weeks. These mice or rats are sacrificed at 6-24 months of age for analyses of Aβ levels and Aβ load in the brain. Quantitative Aβ image analysis is performed using anti-β-Amyloid (clone 4G8).

To measure spatial working memory and exploratory activity, animals are individually placed in one arm of a radially symmetric Y-maze made of opaque gray acrylic or other suitable maze for behavioral testing. The RAWM test or a suitable test is conducted for mouse or rat behavior evaluation.

### Example 15: Efficacy Study of TfR-MINC-Trastuzumab, Tet1-MINC-Trastuzumab, TC13-MINC-Trastuzumab and Adenosine-MINC-Trastuzumab in Glioma Mouse Model (Prophetic Example)

### Objective

This experiment is intended to demonstrate the therapeutic efficacy of TfR, Tet1, TC13 peptide and adenosine conjugation in treating glioma. in vivo imaging system (IVIS) is used to measure tumor volume.

### Materials

TfR-MINC-trastuzumab nanoparticles, Tet1-MINC-trastuzumab nanoparticles, TC13-MINC-trastuzumab nanoparticles, adenosine-MINC-trazstuzumab and MINC-trastuzumab nanoparticles are prepared using the method taught in Example 4.

Trastuzumab is purchased from EirGenix.

A172 glioma cell line is from ATCC (CRL1620).

D-Luciferin is purchased from Sigma-Aldrich.

### Method

An orthotopic glioma mouse model is used to evaluate the efficacy of TfR-MINC-trastuzumab nanoparticles, Tet1-MINC-trastuzumab nanoparticles, TC13-MINC-trastuzumab nanoparticles, adenosine-MINC-trastuzumab and MINC-trastuzumab nanoparticles in treating glioma. For imaging tumor size, A172 cell line is engineered with luciferase genes (A172-Luc). For the generation of the mouse model, a skull burr hole is created in the right frontal brain area and then, 1×10⁶ A172-Luc cells (suspended in 3 l of DMEM) are injected slowly to the mouse brain. Two weeks after tumor implantation, TfR-MINC-trastuzumab, Tet1-MINC-trastuzumab, TC13-MINC-trastuzumab, adenosine-MINC-trastuzumab and MINC-trastuzumab are i.v. injected at 0.1 to 10 mg/kg twice per week for 5-10 weeks. Tumor size is examined using an in vivo imaging system (IVIS) biweekly. After the mice are anesthetized, mice are injected with luciferase substrate solution intraperitoneally and then transferred to the IVIS chamber for image acquisition.

### LIST OF ABBREVIATIONS

ADO Adenosine
HENECA N-Ethylcarboxamidoadenosine
GDNF Glial Cell Line-Derived Neurotrophic Factor
PDGF Platelet-Derived Growth Factor
CDNF Cerebral Dopamine Neurotrophic Factor
TfR Transferrin Receptor
IR Insulin Receptor
LDLR Low-Density Lipoprotein Receptor
Dhh Desert Hedgehog
P75NTR p75 Neurotrophin Receptor
NCAM Neural Cell Adhesion Molecule
PDGFRA Platelet-Derived Growth Factor Receptor Alpha
NG2 Neuron-Glia Antigen 2
MOG Myelin Oligodendrocyte Glycoprotein
MBP Myelin Basic Protein
TMEM119 Transmembrane Protein 119
CX3CR1 CX3C Chemokine Receptor 1
CD45 Cluster of Differentiation 45

What is claimed is:

1. A conjugate comprising:
(a) a CNS-targeting ligand, (b) a hydrophilic polymer of polyethylene glycol (PEG), polylactic acid (PLA), polylactic-co-glycolic acid (PLGA), or dextran, and (c) a flavonoid of EGCG, EC, EGC, or ECG, as shown in the structures below: 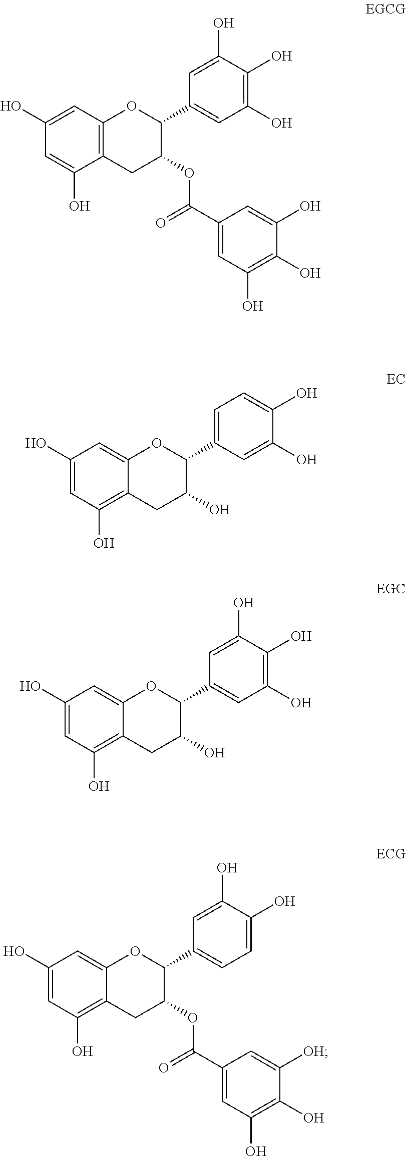 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
wherein the hydrophilic polymer covalently binds to the flavonoid and the CNS-targeting ligand,
wherein the CNS-targeting ligand is selected from the group consisting of:
TfR peptide having the amino acid sequence of THRPPMWSPVWP (SEQ ID NO: 1),
Tet1 peptide having the amino acid sequence of HLNILSTLWKYRC (SEQ ID NO: 2),
TC13 peptide having the amino acid sequence of TGNYKALHPHNGC (SEQ ID NO: 3),
apamin peptide having the amino acid sequence of CNCKAPETALCARRCQQH (SEQ ID NO: 4),
regulon polypeptides having the amino acid sequence of PTVIHGKREVTLHL (SEQ ID NO: 5),
RAP peptide having the amino acid sequence of ELKHFEAKIEKHNHYQKQLE (SEQ ID NO: 6),
Angiopep-2 peptide having the amino acid sequence of TFFYGGSRGKRNNFKTEEY (SEQ ID NO: 7),
TAT peptide having the amino acid sequence of GGGGYGRKKRRQRRR (SEQ ID NO: 8),
SynB1 peptide having the amino acid sequence of RGGRLSYSRRRFSTSTGR (SEQ ID NO: 9),
Leptin 30 peptide having the amino acid sequence of YQQVLTSLPSQNVLQIANDLENLRDLLHLLC (SEQ ID NO: 10),
LNP peptide having the amino acid sequence of KKRTLRKNDRKKRC (SEQ ID NO: 11),
ApoB peptide having the amino acid sequence of SSVIDALQYKLEGTTRLTRKRGLKLATALSLSNKFVEGS (SEQ ID NO: 12),
RVG-29 peptide having the amino acid sequence of YTIWMPENPRPGTPCDIFTNSRGKRASNG (SEQ ID NO: 13),
T7 peptide having the amino acid sequence of HAIYPRH (SEQ ID NO: 14),
GSH peptide having the amino acid sequence of ECG,
CRT peptide having the amino acid sequence of CRTIGPSVC (SEQ ID NO: 15),
CAQK peptide having the amino acid sequence of CAQK (SEQ ID NO: 16),
TACL05 peptide having the amino acid sequence of SACPSHLTKMCGGG (SEQ ID NO: 17), and
adenosine, 5′-N-ethylcarboxamidoadenosine, glutamic acid or glutamate glutamate, and γ-aminobutyric acid (GABA).

2. The conjugate of claim 1, wherein the flavonoid is EGCG.

3. The conjugate of claim 1, wherein the hydrophilic polymer is PEG.

4. The conjugate of claim 1, wherein the flavonoid is EGCG and the hydrophilic polymer is PEG.

5. A nanoparticle composition comprising nanoparticles having: (a) an outer shell comprising the conjugate of claim 1, optionally (b) an inner shell comprising one or more flavonoid oligomer, and (c) a drug encapsulated within the shells, wherein the flavonoid is EGCG, EC, EGC, or ECG, and the drug is effective to treat a CNS disease.

6. The nanoparticle composition of claim 5, wherein the flavonoid in the conjugate is EGCG.

7. The nanoparticle composition of claim 5, wherein the hydrophilic polymer in the conjugate is PEG.

8. The nanoparticle composition of claim 5, wherein the flavonoid is EGCG and the hydrophilic polymer is PEG, in the conjugate.

9. The nanoparticle composition of claim 5, wherein the encapsulated drug is anti-CD3, anti-CD4, anti-IL-17, anti-CD19, anti-CD20, anti-CD38, anti-β amyloid, anti-tau, anti-α-synuclein, anti-mHtt, anti-IL6R, anti-IL-1β, anti-TREM2, BDNF, CDNF, GDNF, NRTN, PDGF-BB, anti-HER2, anti-EGFR, anti-PD-1, anti-PD-L1, anti-PDGFRA, anti-VEGF, anti-VEGFR2, IL-2, IL-4, IL-12, IFN-α, IFN-β, IFN-γ, or TNF-α.

10. The nanoparticle composition of claim 5, wherein the outer shell further comprises a bare hydrophilic polymer-flavonoid conjugate that does not covalently bind to the CNS-targeting ligand.

11. A method for treating a CNS disease, comprising the step of administering to a subject in need thereof an effective amount of the nanoparticle composition of claim 5.

12. The method of claim 11, wherein the CNS disease is Alzheimer's disease, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-β amyloid, anti-tau, anti-IL6R, anti-IL-1β, anti-CD38, anti-TREM2, GDNF, NRTN, PDGF-BB, CDNF, or BDNF.

13. The method of claim 11, wherein the CNS disease is Parkinson's disease, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-α-synuclein, anti-IL6R, anti-IL-1β, anti-CD38, anti-TREM2, GDNF, NRTN, PDGF-BB, CDNF, or BDNF.

14. The method of claim 11, wherein the CNS disease is Lewy body dementia, and the drug is anti-CD3, anti-CD33, anti-CD36, anti-CD39, anti-CD73, anti-PD-1, anti-PD-L1, anti-PD-L2, anti-CTLA4, anti-GZM-A, anti-GZM-B, anti-TAM, anti-FcγRI, anti-RAGE, anti-APOE, anti-CR1, anti-NLRP3, anti-β amyloid or, anti-α-synuclein, anti-IL6R, anti-IL-1β, anti-CD38, anti-TREM2, GDNF, NRTN, PDGF-BB, CDNF, or BDNF.

15. The method of claim 11, wherein the CNS disease is brain tumor, and the drug is doxorubicin, disulfiram, celecoxib, temsirolimus, everolimus, vorinostat, cabozantinib, marizomib, fimepinostat, acetazolamide, metformin, vinblastine, cyclophosphamide, anti-HER2, anti-EGFR, anti-PD-1, anti-PD-L1, anti-PDGFRA, anti-VEGF, anti-VEGFR2, IL-2, IL-4, IL-12, IFN-α, IFN-β, IFN-γ, or TNF-α.
